(19) United States
(12) Patent Application Publication
(10) Pub. No.: US 2008/0161574 A1
(43) Pub. Date: Jul. 3, 2008

(54) DIAZAFLUORENE COMPOUND

(75) Inventors: Hiroki Ohrui, Kawasaki-shi (JP); Akihiro Senoo, Kawasaki-shi (JP); Tetsuya Kosuge, Kawasaki-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/960,125

(22) Filed: Dec. 19, 2007

(30) Foreign Application Priority Data
Dec. 27, 2006 (JP) 2006-351758

(51) Int. Cl. C07D 471/04

(52) U.S. Cl. 546/85

(57) ABSTRACT

The present invention provides a novel diazafluorene compound for manufacturing a 4,5-diazafluorene derivative. The diazafluorene compound is represented by the general formula (1):

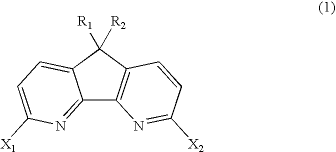

wherein R₁ and R₂ each represent a hydrogen atom, a substituted or unsubstituted alkyl group, or a substituted or unsubstituted aryl group, and may be the same or different from each other; and X₁ and X₂ each represent a halogen atom, and may be the same or different from each other.

### BACKGROUND OF THE INVENTION

1. Field of the Invention

The present invention relates to a novel diazafluorene compound.

2. Description of the Related Art

Because a compound having a diazafluorene skeleton contains a nitrogen-containing aromatic heterocyclic ring, such a compound can obtain stable amorphous film properties and exhibits excellent electron transporting properties. Based on these properties, such a compound can be used as a charge transporting material for an electrophotographic photosensitive member, an organic electroluminescence device, a photoelectric transducer, an organic semiconductor device, and an organic solar cell. Moreover, such a compound, when applied to an organic electroluminescence device, can contribute to achieving high light emitting efficiency and lowering the voltage of the device, and therefore, the compound is suitable for a material for an organic electroluminescence device. Chem. Lett. 33, 276 (2004) and Org. Lett. 7, 1979, (2005) disclose, as a specific example of application of a material for an organic electroluminescence device, that a compound having a 4,5-diazafluorene skeleton in which the 3- and 6-positions are not substituted is applied to an electron transporting light emitting layer or a hole blocking layer.

For example, Japanese Patent Application Laid-Open No. H07-503006, Japanese Patent Application Laid-Open No. 2003-77670, Japanese Patent Application Laid-Open No. 2004-91444, and International Publication No. WO 2005/123634 refer to the compound having the 4,5-diazafluorene skeleton. Japanese Patent Application Laid-Open No. H07-503006 discloses utilizing a compound having an aralkyl group at the 9-position and has a halogen atom at the 8-position of the diazafluorene skeleton as a neurological dysfunction therapeutic agent. Japanese Patent Application Laid-Open No. 2003-77670, Japanese Patent Application Laid-Open No. 2004-91444, and International Publication No. WO 2005/123634 are mentioned as an example of an organic electroluminescence device utilizing the compound having the 4,5-diazafluorene skeleton. A compound having a substituent at the 2- or 7-position of the diazafluorene skeleton is mainly used. However, in order to increase the thermal stability of the compound, a 4,5-diazafluorene compound having a substituent at the 3- or 6-position which is adjacent to a nitrogen atom is more preferable.

The only production process for introducing a substituent to the 3- or 6-position has heretofore been one involving subjecting a nucleophilic reaction of a lithium compound to the 4,5-diazafluorene compound, in which the 3- or 6-position is not substituted as described in Japanese Patent Application Laid-Open No. 2004-91444. However, in this production process, because an extremely low reaction temperature is required and a lithium compound which is an antiposic reagent is used, there is a limitation on industrially obtaining the 4,5-diazafluorene compound having a substituent at the 3- or 6-position. Furthermore, whether or not the production process can be utilized depends on the production propriety of the lithium compound to be utilized or its solubility. Therefore, the types of a substituent to be introduced to the 3rd or 6th position of a diazafluorene skeleton are limited.

### SUMMARY OF THE INVENTION

The present invention has been made in order to solve the above-mentioned problems of the conventional technology. The present invention aims to provide a novel diazafluorene compound for industrially manufacturing a 4,5-diazafluorene derivative.

The present inventors carried out extensive research so as to achieve the object, and, as a result, the present invention has been accomplished.

That is, the present invention provides a diazafluorene compound represented by the general formula (1):

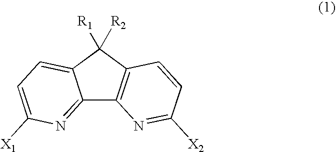

where R₁ and R₂ each represent a hydrogen atom, a substituted or unsubstituted alkyl group, or a substituted or unsubstituted aryl group, and may be the same or different from each other; and X₁ and X₂ each represent a halogen atom, and may be the same or different from each other.

The present invention can provide a novel diazafluorene compound for industrially manufacturing a 4,5-diazafluorene derivative.

### DESCRIPTION OF THE EMBODIMENTS

First, the diazafluorene compound of the present invention will be described.

The diazafluorene compound of the present invention is represented by the general formula (1).

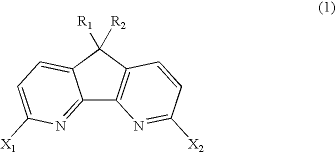

In Formula (1), R₁ and R₂ each represent a hydrogen atom, a substituted or unsubstituted alkyl group, or a substituted or unsubstituted aryl group.

Examples of the alkyl group represented by R₁ and R₂ include, but of course are not limited to, a methyl group, an ethyl group, an n-propyl group, an n-butyl group, an n-pentyl group, an n-hexyl group, an n-heptyl group, an n-octyl group, an n-decyl group, an iso-propyl group, an iso-butyl group, a sec-butyl group, a tert-butyl group, an iso-pentyl group, a neopentyl group, a tert-octyl group, a fluoromethyl group, a difluoromethyl group, a trifluoromethyl group, a 2-fluoroethyl group, a 2,2,2-trifluoroethyl group, a perfluoroethyl group, a 3-fluoropropyl group, a perfluoropropyl group, a 4-fluorobutyl group, a perfluorobutyl group, a 5-fluoropentyl group, a 6-fluorohexyl group, a chloromethyl group, a trichloromethyl group, a 2-chloroethyl group, a 2,2,2-trichloroethyl group, a 4-chlorobutyl group, a 5-chloropentyl group, a 6-chlorohexyl group, a bromomethyl group, a 2-bromoethyl group, an iodomethyl group, a 2-iodoethyl group, a hydroxymethyl group, a hydroxyethyl group, a cyclopropyl group, a cyclobutyl group, a cyclopentyl group, a cyclohexyl group, a cyclopentylmethyl group, a cyclohexylmethyl group, a cyclohexylethyl group, a 4-fluorocyclohexyl group, a norbornyl group, and an adamantyl group.

Examples of the aryl group represented by R₁ and R₂ include, but of course are not limited to, a phenyl group, a 4-methylphenyl group, a 4-methoxyphenyl group, a 4-ethylphenyl group, a 4-fluorophenyl group, a 4-trifluorophenyl group, a 3,5-dimethylphenyl group, a 2,6-diethylphenyl group, a mesityl group, a 4-tert-butylphenyl group, a ditolylaminophenyl group, and a biphenyl group.

Examples of substituents which may be substituted for the alkyl groups and the aryl groups include, but are of course not limited to: alkyl groups such as a methyl group, an ethyl group, a propyl group, and a trifluoromethyl group; aryl groups such as a phenyl group and a biphenyl group; heterocyclic groups such as a thienyl group and a pyrrolyl group; substituted amino groups such as a dimethylamino group, a diethylamino group, a dibenzylamino group, a diphenylamino group, a ditolylamino group, and a dianisolylamino group; alkoxy groups such as a methoxy group and an ethoxy group; halogen atoms such as fluorine, chlorine, bromine, and iodine; hydroxyl groups; cyano groups; and nitro groups.

R₁ and R₂ may be the same or different from each other.

In Formula (1), X₁ and X₂ each represent a halogen atom.

Examples of the halogen atom represented by X₁ and X₂ include fluorine, chlorine, bromine, and iodine.

X₁ and X₂ may be the same or different from each other.

The diazafluorene compound of the present invention is produced by the following steps. First, peroxide is acted, under an argon stream, to a compound represented by the general formula (2):

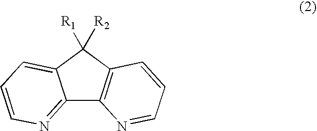

where R₁ and R₂ are the same as those of Formula (1), thereby converting the compound to a compound represented by the general formula (3):

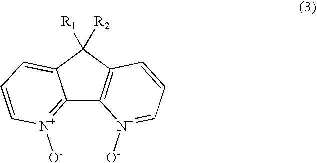

where R₁ and R₂ are the same as those of Formula (1).

The solvent used for this reaction is an organic solvent such as chloroform, methylene chloride, toluene, or dioxane. The weight of the solvent to be used is 5 times or more to 50 times or less, and preferably 10 times or more to 20 times or less with respect to the weight of the compound represented by Formula (2).

The peroxide to be used for this reaction is hydrogen peroxide, peracetic acid, meta-chloroperbenzoic acid, and perbenzoic acid. The weight of the peroxide to be used is 1 Eq or more to 10 Eq or less, and preferably 1 Eq or more to 5 Eq or less, based on the number of moles of the compound represented by Formula (2).

The temperature during this reaction is 0° C. or higher to 100° C. or lower, and preferably 20° C. or higher to 40° C. or lower.

Then, a halogenating agent is acted, under an argon stream, to the compound represented by Formula (3), thereby obtaining the diazafluorene compound of the present invention represented by Formula (1).

As the solvent used for this reaction, toluene, dioxane, N,N-dimethylformamide, and triethylamine can be mentioned. A halogenating agent mentioned later may be used as a direct solvent. The weight of the solvent to be used is 2 times or more to 50 times or less, and preferably 10 times or more to 20 times or less with respect to the weight of the compound represented by Formula (3).

As the halogenating agent to be used for this reaction, phosphorus oxychloride, phosphorus pentachloride, oxyphosphorus bromide, phosphorus pentabromide, triphenylphosphine/N-succinimide chloride, triphenylphosphine/N-succinimide bromide, and triphenylphosphine/N-iodination succinimide can be used.

The temperature during this reaction is 10° C. or higher to 200° C. or lower, and preferably 80° C. or higher to 150° C. or lower.

Hereinafter, specific structural formulae of the diazafluorene compound of the present invention will be shown. However, these formulae are merely typical examples thereof and the present invention is not limited to the following formulae.

### COMPOUND EXAMPLE 1

In the general formula (1), X₁ and X₂ each represent a chlorine atom, and R₁ and R₂ each represent a hydrogen atom or an alkyl group such as a methyl group, an ethyl group, or a trifluoromethyl group.

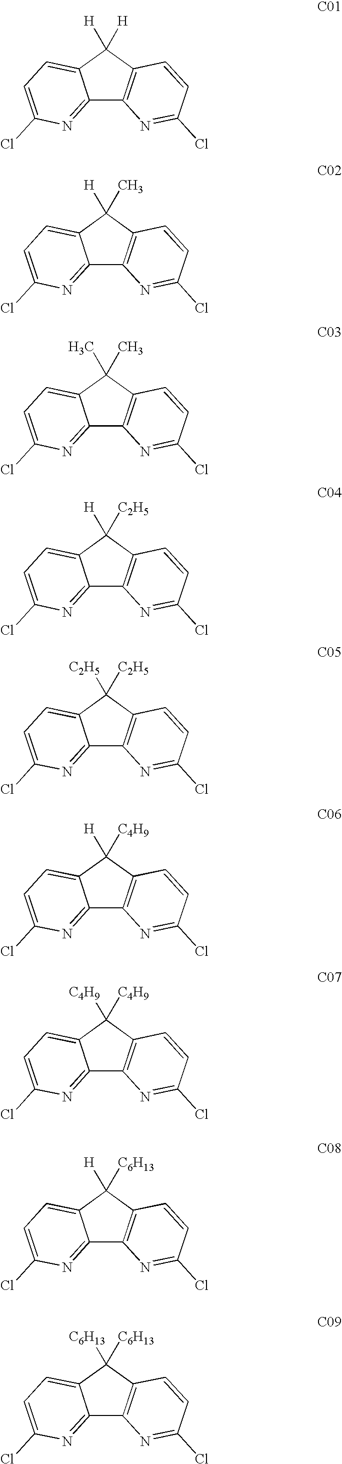

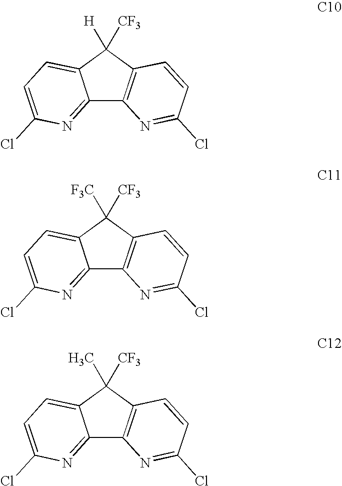

### COMPOUND EXAMPLE 2

In the general formula (1), X₁ and X₂ represent a chlorine atom, and R₁ and R₂ represent an aryl group such as a phenyl group or a tolyl group.

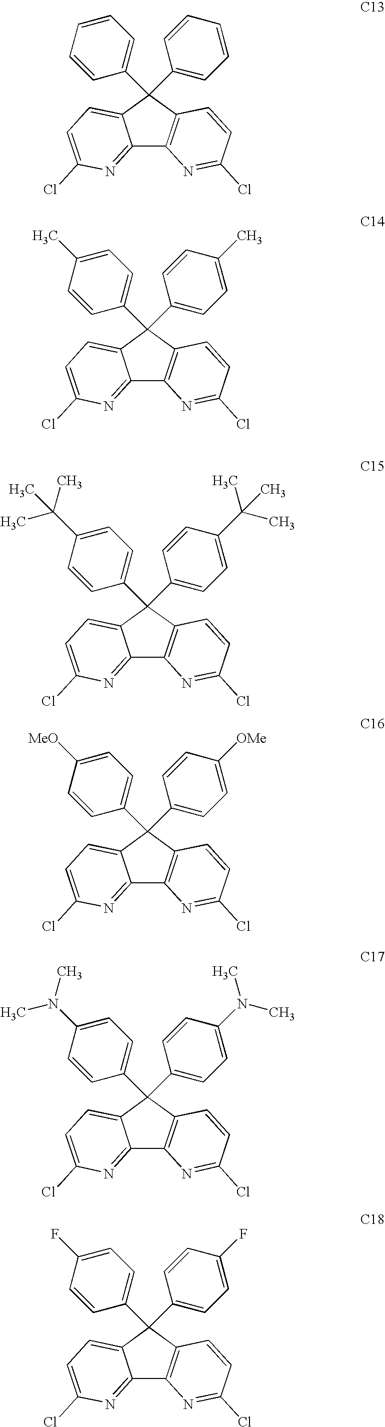

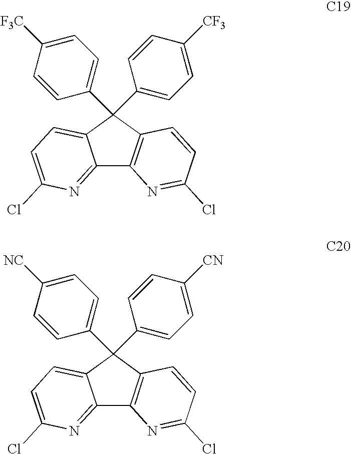

### COMPOUND EXAMPLE 3

In the general formula (1), X₁ and X₂ each represent a bromine atom, and R₁ and R₂ each represent a hydrogen atom or an alkyl group such as a methyl group, an ethyl group, or a trifluoromethyl group.

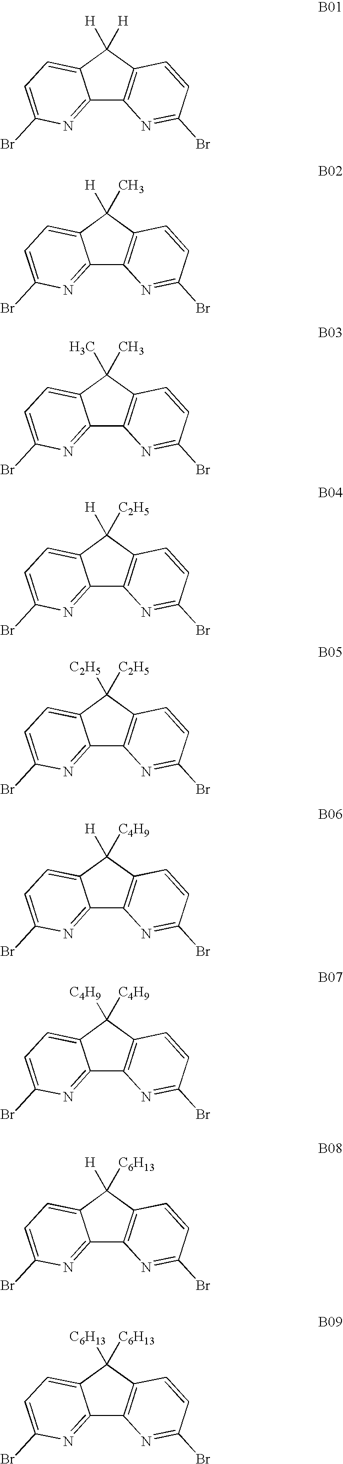

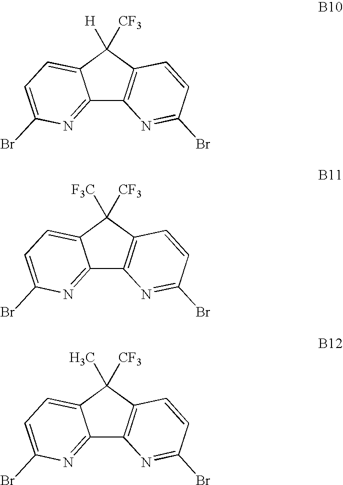

### COMPOUND EXAMPLE 4

In the general formula (1), X₁ and X₂ represent a bromine atom, and R₁ and R₂ represent an aryl group such as a phenyl group or a tolyl group.

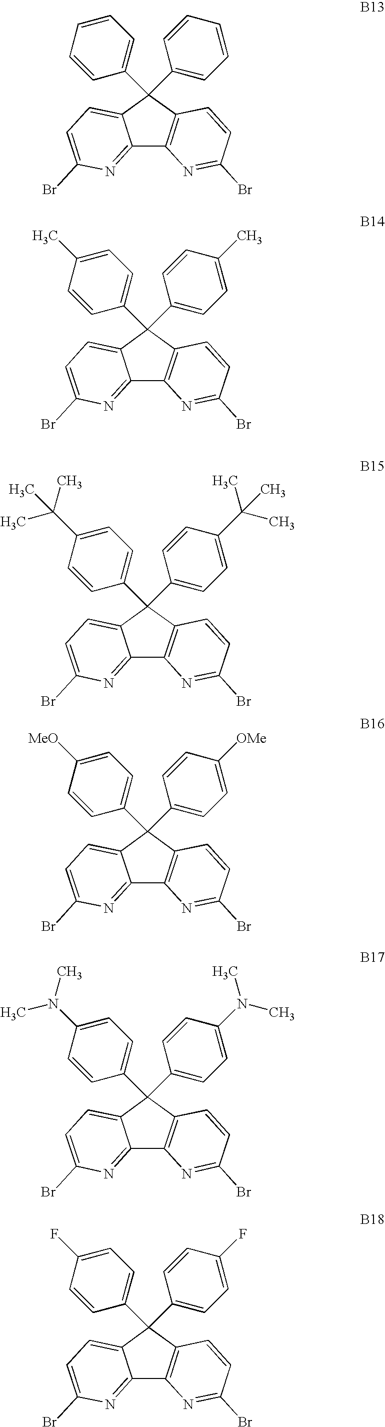

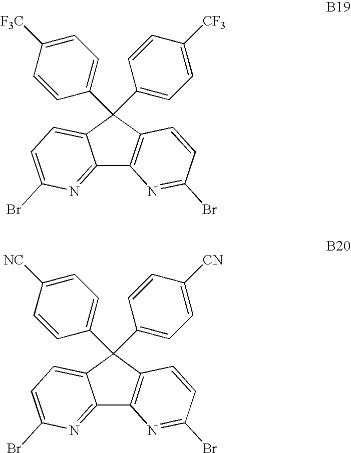

### COMPOUND EXAMPLE 5

In the general formula (1), X₁ and X₂ each represent an iodine atom, and R₁ and R₂ each represent a hydrogen atom or an alkyl group such as a methyl group, an ethyl group, or a trifluoromethyl group.

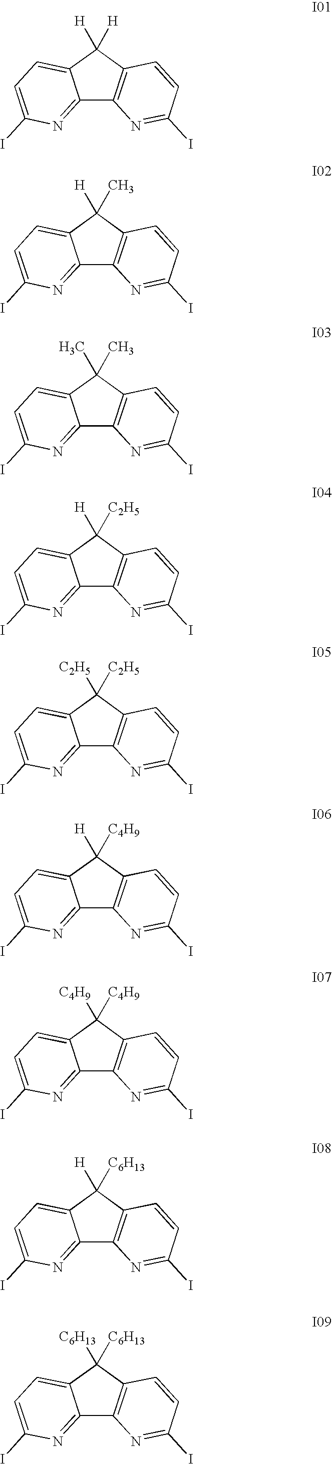

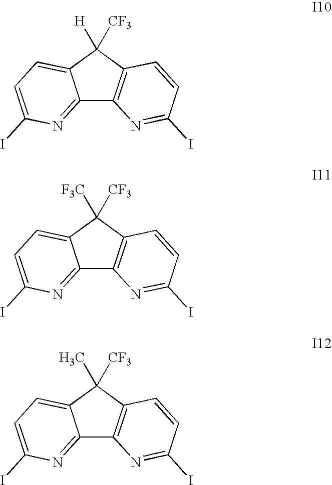

### COMPOUND EXAMPLE 6

In the general formula (1), X₁ and X₂ represent an iodine atom, and R₁ and R₂ represent an aryl group such as a phenyl group or a tolyl group.

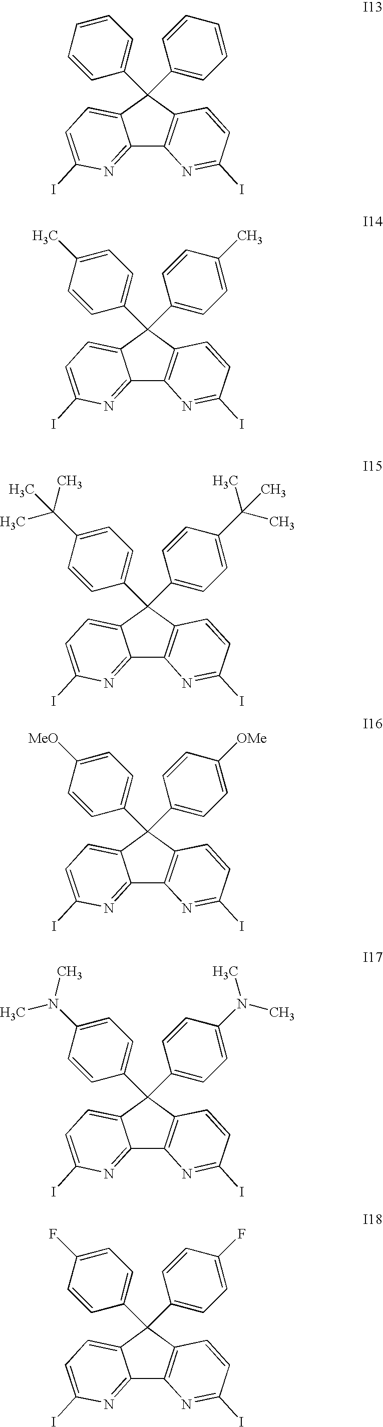

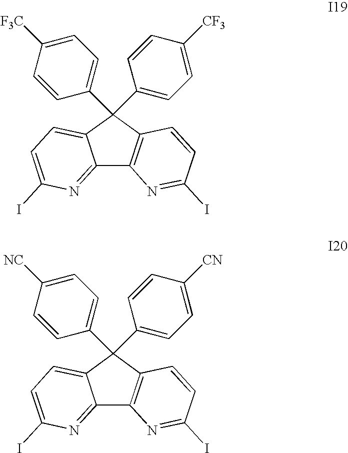

Next, a production process of a 4,5-diazafluorene derivative will be described. The production process of the 4,5-diazafluorene derivative is performed by subjecting the diazafluorene compound of the present invention and an organic boronic acid compound or an organic boronic acid ester to a condensation reaction in the presence of a transition metal. Thus, the 4,5-diazafluorene derivative represented by the general formula (4) can be obtained.

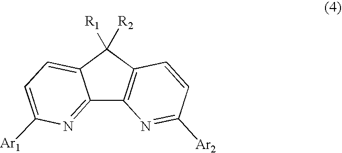

In Formula (4), R₁ and R₂ each represent a hydrogen atom, a substituted or unsubstituted alkyl group, or a substituted or unsubstituted aryl group.

An alkyl group, an aryl group, and a substituent which may be substituted to an alkyl group and an aryl group, represented by R₁ and R₂, are the same as those of R₁ and R₂ of Formula (1)

R₁ and R₂ may be the same or different from each other.

In Formula (4), Ar₁ and Ar₂ each represent a substituted or unsubstituted aryl group, a substituted or unsubstituted heterocyclic group, a substituted or unsubstituted condensed polycyclic aromatic group, or a substituted or unsubstituted condensed polycyclic heterocyclic group.

Examples of the aryl group represented by Ar₁ and Ar₂ include, but of course are not limited to, a phenyl group, a 4-methylphenyl group, a 4-methoxyphenyl group, a 4-ethylphenyl group, a 4-fluorophenyl group, a 4-trifluorophenyl group, a 3,5-dimethylphenyl group, a 2,6-diethylphenyl group, a mesityl group, a 4-tert-butylphenyl group, a ditolylaminophenyl group, and a biphenyl group.

Examples of the heterocyclic group represented by Ar₁ and Ar₂ include, but of course are not limited to, a pyridyl group, a pyrrolyl group, a bipyridyl group, a methylpyridyl group, a pyrimidinyl group, a pyrazinyl group, a pyridazinyl group, a terpyrrolyl group, a thienyl group, a terthienyl group, a propylthienyl group, a furyl group, an oxazolyl group, an oxadiazolyl group, a thiazolyl group, and a thiadiazolyl group.

Examples of the condensed polycyclic aromatic group represented by Ar₁ and Ar₂ include, but of course are not limited to, a naphthyl group, an acenaphthylenyl group, an anthrylgroup, a phenan tolyl group, a pyrenyl group, an acephenantrilenyl group, aceanetrilenyl group, a crycenyl group, a dibenzocrycenyl group, a benzoanthrylgroup, a dibenzo anthrylgroup, a napthacenyl group, a picenyl group, a pentacenyl group, a fluorenyl group, a 9,9-dihydroanthryl group, a triphenyl group, a perilenyl group, a fluoranethenyl group, and a benzofluoranethenyl group.

Examples of the condensed polycyclic heterocyclic group represented by Ar₁ and Ar₂ include, but of course are not limited to, a quinolyl group, an isoquinolyl group, a benzothienyl group, a dibenzothienyl group, a benzofuryl group, an isobenzofuryl group, a dibenzofuryl group, a quinoxalinyl group, a naphthylidinyl group, a quinazolinyl group, a phenantridinyl group, an indolidinyl group, a phenadinyl group, a carbazolyl group, an acridinyl group, a phenadinyl group, and a diazafluorenyl group.

Examples of substituents which may be substituted for the aryl groups, the heterocyclic groups, the condensed polycyclic aromatic groups, and the heterocyclic groups include, but of course are not limited to: alkyl groups such as a methyl group, an ethyl group, a propyl group, and a trifluoromethyl group; aryl groups such as a phenyl group and a biphenyl group; heterocyclic groups such as a thienyl group and a pyrrolyl group; amino groups such as a dimethylamino group, a diethylamino group, a dibenzylamino group, a diphenylamino group, a ditolylamino group, and a dianisolylamino group; alkoxy groups such as a methoxy group and an ethoxy group; halogen atoms such as fluorine, chlorine, bromine, and iodine; hydroxyl groups; cyano groups; and nitro groups.

Ar₁ and Ar₂ may be the same or different from each other.

Specifically, the production process of the 4,5-diazafluorene derivative is a process involving subjecting the diazafluorene compound of the present invention and the organic boronic acid compound or the organic boronic acid ester represented by the following general formula (5) or (6) to a condensation reaction in the presence of a transition metal catalyst.

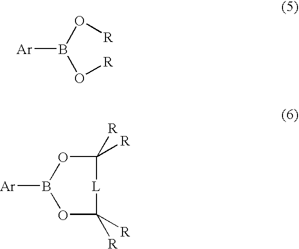

In Formula (5), R represents a hydrogen atom or a substituted or unsubstituted alkyl group, and Ar is the same as that of Ar₁ and Ar₂ of Formula (4).

In Formula (6), R is the same as that of Formula (5). Ar is the same as Ar₁ and Ar₂ of Formula (4). L represents a single bond or methylene.

Examples of the alkyl group represented by R include, but of course are not limited to, a methyl group, an ethyl group, an n-propyl group, an n-butyl group, an n-pentyl group, an n-hexyl group, an n-heptyl group, an n-octyl group, an n-decyl group, an iso-propyl group, an iso-butyl group, a sec-butyl group, a tert-butyl group, an iso-pentyl group, a neopentyl group, a tert-octyl group, a fluoromethyl group, a difluoromethyl group, a trifluoromethyl group, a 2-fluoroethyl group, a 2,2,2-trifluoroethyl group, a perfluoroethyl group, a 3-fluoropropyl group, a perfluoropropyl group, a 4-fluorobutyl group, a perfluorobutyl group, a 5-fluoropentyl group, a 6-fluorohexyl group, a chloromethyl group, a trichloromethyl group, 2-chloroethyl group, a 2,2,2-trichloroethyl group, a 4-chlorobutyl group, a 5-chloropentyl group, a 6-chlorohexyl group, a bromomethyl group, a 2-bromoethyl group, an iodomethyl group, a 2-iodoethyl group, a hydroxymethyl group, a hydroxyethyl group, a cyclopropyl group, a cyclobutyl group, a cyclopentyl group, a cyclohexyl group, a cyclopentylmethyl group, a cyclohexylmethyl group, a cyclohexylethyl group, a 4-fluorocyclohexyl group, a norbornyl group, and an adamantyl group.

Examples of substituents which may be substituted for the alkyl groups include, but of course are not limited to: alkyl groups such as a methyl group, an ethyl group, a propyl group, and a trifluoromethyl group; aryl groups such as a phenyl group and a biphenyl group; heterocyclic groups such as a thienyl group and a pyrrolyl group; amino groups such as a dimethylamino group, a diethylamino group, a dibenzylamino group, a diphenylamino group, a ditolylamino group, and a dianisolylamino group; alkoxy groups such as a methoxy group and an ethoxy group; halogen atoms such as fluorine, chlorine, bromine, and iodine; hydroxyl groups; cyano groups; and nitro groups.

As the solvent used for the condensation reaction, toluene, xylene, dioxane, N,N-dimethylformamide, ethanol, and water can be mentioned, and they may be used in combination. The weight of the solvent to be used is 5 times or more to 100 times or less, and preferably 10 times or more to 20 times or less with respect to the weight of the compound represented by the general formula (1).

The transition metal catalyst used for this condensation reaction is zerovalent or divalent palladium such as tetrakis triphenylphosphine palladium (0), dichlorobis triphenylphosphine palladium (2), palladium acetate (2), and bis(dibenzylideneacetone) palladium. The number of equivalent of the transition metal catalyst is 0.1 mol % or more to 80 mol % or less, and preferably 5 mol % or more to 20 mol % or less based on the number of moles of the compound represented by the general formula (1).

The temperature during this condensation reaction is 10° C. or higher to 200° C. or lower, and preferably 80° C. or higher to 150° C. or lower.

According to this method, the control of the reaction temperature is easier compared with a conventional process using a lithium compound and the generation of a by-product can be inhibited. Therefore, the 4,5-diazafluorene derivative represented by Formula (4) can be obtained industrially and simply.

Hereinafter, the present invention will be described more specifically with reference to Examples, but is not limited thereto.

### EXAMPLE 1

(Production Process of Exemplified compound No. C03)

The exemplified compound C03 of the present invention was produced by a method described below.

(1) Synthesis of 4,5-diazafluorenone (intermediate compound 1)

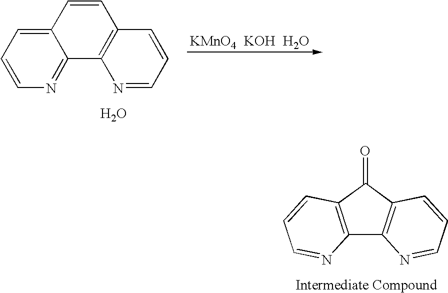

3.4 kg (21.5 mol) of potassium permanganate and 54.7 kg of water were stirred at 30° C. overnight, to form an aqueous solution. Next, 1.3 kg (6.57 mol) of 1,10-phenanthroline monohydrate, 102.6 kg of water, and 1.36 kg of potassium hydroxide were stirred under heat at 90° C. Then, the aqueous potassium permanganate solution prepared in advance was added dropwise in the resultant at a temperature in the range of ₉₀° C. or higher to 94° C. or lower. After the completion of the dropwise addition, the resultant was stirred at 95° C. for 30 minutes, and then cooled to a temperature in the range of 40° C. or higher to 50° C. or lower. 15 L of warm water was added to the resultant, and the mixture was subjected to celite filtration. Then, 16.5 kg of methylene chloride was added to the filtrate, and then extracted. The aqueous layer was extracted twice with 16.5 kg of methylene chloride, and the organic layer was concentrated after mirabilite desiccation. Crude crystals were washed with 3 L acetone to remove slurry, thereby obtaining 550 g of 4,5-diazafruorenone (intermediate compound 1).

(2) Synthesis of 9H-4,5-diazafluorene (intermediate compound 2)

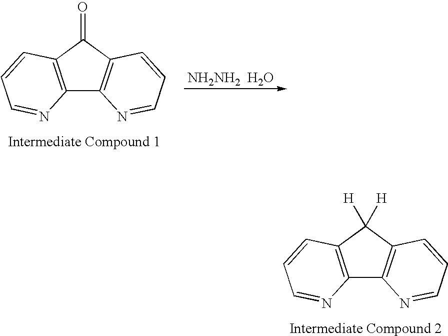

550 g (3.02 mol) of 4,5-diazafluorenone (intermediate compound 1) was dissolved in 5.5 L of diethylene glycol under a nitrogen atmosphere. Next, 3.85 L (79.4 mol) of hydrazine monohydrate was added to this solution at room temperature, and the temperature was increased up to 98° C. Then, the mixture was heated under stirring for 5 hours. After the completion of the reaction, the temperature was cooled to 10° C., and then 11 L of water was added. 18 kg of methylene chloride was added to the resultant, and then extracted. Then, the aqueous layer was extracted again with 18 kg of methylene chloride, and then the organic layer was concentrated. A crude material obtained after the concentration was purified by column chromatography (silica gel: 11 kg, Mobile phase: methylene chloride/ethyl acetate=1/5 followed by ethyl acetate), thereby obtaining 204 g of 9H-4,5-diazafluorene (intermediate compound 2).

(3) Synthesis of 9,9-dimethyl-9H-4,5-diazafluorene (intermediate compound 3)

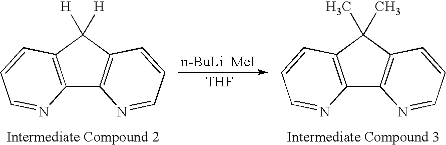

204 g (1.21 mol) of 9H-4,5-diazafluorene (intermediate compound 2) was dissolved in 14 L of tetrahydrofuran under an Ar stream. This solution was cooled to −48° C., and then 930 mL (1.47 mol) of hexane solution (concentration: 1.58 mol/L) of normal butyllithium was added dropwise at a temperature in the range of −48° C. or higher to −42° C. or lower over 2 hours 30 minutes, followed by stirring for 1 hour. Next, 206 g (1.45 mol) of iodomethane was added dropwise over 30 minutes at a temperature in the range of −35° C. or higher to −25° C. or lower, and the resultant was stirred for 2 hours. Thereafter, 930 mL (1.47 mol) of hexane solution (concentration: 1.58 mol/L) of normal butyllithium was added dropwise at a temperature in the range of −45° C. or higher to −42° C. or lower over 1 hour 20 minutes, followed by stirring for 1 hour. Next, 206 g (1.45 mol) of methyl iodide was added dropwise over 20 minutes at a temperature in the range of −38° C. or higher to −35° C. or lower, and then the resultant was stirred overnight while increasing the temperature up to room temperature. Water was added to the resultant to stop the reaction, and the mixture was extracted with ethyl acetate. Thereafter, the organic layer was washed with an aqueous 10% sodium sulfite solution and a saturated sodium chloride solution in this order, and then the resultant was concentrated after mirabilite desiccation. The obtained crude material was purified by column chromatography (silica gel: 10 kg, Mobile phase: ethyl acetate/hexane=8/2 followed by 9/1), thereby obtaining 109 g of 9,9-dimethyl-9H-4,5-diazafluorene (intermediate compound 3).

195.10 which is M+ of this compound was confirmed by matrix-assisted laser desorption-ionization time-of-flight mass spectrometry (MALDI-TOF MS).

¹H-NMR(CDCl₃):δ(ppm)=8.68(dd,2H,J1=4.8,J2=1.2 Hz), 7.76(dd,2H,J1=7.6,J2=1.2 Hz),7.28(dd,2H,J1=7.6,J2=4.8 Hz), 1.53(s,6H)

(4) Synthesis of 9,9-dimethyl-9H-4,5-diazafluorene, 4,5-dioxide (intermediate compound 4)

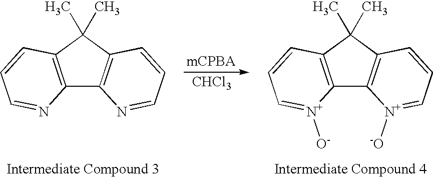

109 g (0.56 mol) of 9,9-dimethyl-9H-4,5-diazafluorene (intermediate compound 3) was dissolved in 1100 mL of chloroform under an Ar stream. 382 g (1.53 mol) of 69% meta-chloroperbenzoic acid was added to this solution, and then the mixture was stirred for 3 hours. Thereafter, it was confirmed that the intermediate compound 3 disappeared, and then the reaction was stopped. After the completion of the reaction, the precipitate was filtered, washed with chloroform, and the filtrate was concentrated after mirabilite desiccation. Chloroform was added to the obtained mixture of an oily substance and a solid matter, and then the resultant was washed to remove slurry and separately the solid matter was removed by filtration. This filtrate was concentrated, and the obtained oily red substance was purified by column chromatography (NH silica gel: 7.5 kg, mobile phase: ethyl acetate/methanol=2/1). Thus, 88.0 g of 9,9-dimethyl-9H-4,5-diazafluorene 4,5-dioxide (intermediate compound 4) was obtained.

227.6 which is M+ of this compound was confirmed by matrix-assisted laser desorption-ionization time-of-flight mass spectrometry (MALDI-TOF MS).

¹H-NMR(CDCl₃):δ(ppm)=8.14(dd,2H,J1=6.4,J2=1.2 Hz), 7.52(dd,2H,J1=7.6,J2=1.2 Hz),7.40(dd,2H,J1=7.6,J2=6.4 Hz), 1.47(s,6H)

(5) Synthesis of exemplified compound No.C03

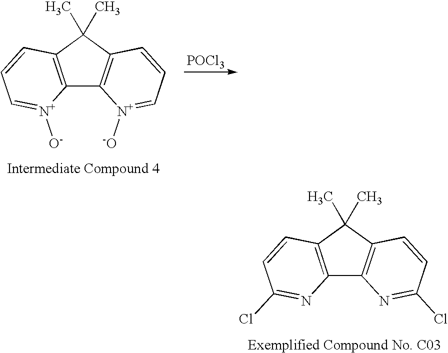

88.0 g (0.39 mol) of 9,9-dimethyl-9H-4,5-diazafluorene 4,5-dioxide (intermediate compound 4) and 900 mL of phosphorus oxychloride were charged under an Ar stream, and stirred under heat overnight at a temperature in the range of 97° C. or higher to 98° C. or lower. After the completion of the reaction, the resultant was cooled to room temperature and phosphorus oxychloride was concentrated under reduced pressure. 200 mL of chloroform was added dropwise to this concentrated liquid, and the mixture was added dropwise in 4 L of sodium bicarbonate water. After being stirred for 1 hour, the resultant was extracted with chloroform, and then the organic layer was washed twice with saturated sodium chloride solution. The resultant was concentrated after mirabilite desiccation. The obtained solid matter was purified by column chromatography (silica gel: 2.2 kg, mobile phase: chloroform/ethyl acetate=50/1) and was washed with ethanol to remove slurry, thereby obtaining 33.0 g of a white crystalline exemplified compound No. C03.

263.6 which is M+ of this compound was confirmed by matrix-assisted laser desorption-ionization time-of-flight mass spectrometry (MALDI-TOF MS).

¹H-NMR(CDCl₃):δ(ppm)=8.22(d,2H,J=8.6), 7.58(d,2H,J=8.6),1.51(s,6H)

### EXAMPLE 2

(Production Process of Exemplified Compound No. C16)

The exemplified compound C16 of the present invention can be produced by a method described below, for example.

(1) Synthesis of 9,9-bis(4-methoxyphenyl)-9H-4,5-diazafluorene (intermediate compound 5)

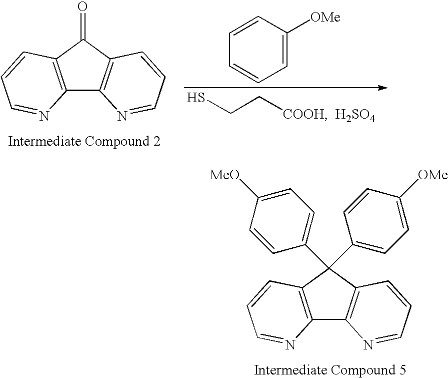

The compounds shown below were charged under a nitrogen stream, and the reaction solution was cooled to 5° C.

Intermediate compound 2: 5.20 g (28.5 mmol)

Anisole: 31 mL (288 mmol)

3-mercaptopropanoic acid: 37 μL (0.295 mmol)

Next, 15 mL of concentrated sulfuric acid was slowly added dropwise to this reaction solution. After the completion of the dropwise addition, this reaction solution was heated up to 70° C. and stirred for 3 hours. Then, methanol was added, whereby the reaction was stopped. An aqueous sodium hydroxide solution was added until the reaction solution became basic. Thereafter, the precipitate was filtered, and washed with water and methanol. The obtained solid matter was purified by washing with acetone to remove slurry, thereby obtaining 8.56 g of 9,9-bis(4-methoxyphenyl)-9H-4,5-diazafluorene (intermediate compound 5).

(2) Synthesis of exemplified compound No. C16

The exemplified compound No. C16 can be synthesized in the same manner as in Example 1 except using the intermediate compound 5 in place of the intermediate compound 3 of Example 1.

Further, the following exemplified compounds can be synthesized in the same manner as in Example 1 except using the compounds described below in place of the phosphorus oxychloride of Example 1.

(Exemplified compound No. B03): Oxyphosphorus bromide

(Exemplified compound No. I03): Triphenylphosphine/N-iodide succinimide

Further, the following exemplified compounds can be synthesized in the same manner as in Example 1 except using the compounds described below in place of the iodomethane of Example 1.

(Exemplified compound No. C05): iodoethane

(Exemplified compound No. C07): iodobuthane

(Exemplified compound No. C09): iodohexane

Further, the following exemplified compounds can be synthesized in the same manner as in Example 2 except using the compounds described below in place of the anisole of Example 2.

(Exemplified compound No. C14): toluene

(Exemplified compound No. C17): N,N-dimethylaniline

### SYNTHESIS EXAMPLE 1

The compound 6 described below was produced using the above-mentioned exemplified compound No. C03. A specific method is described below.

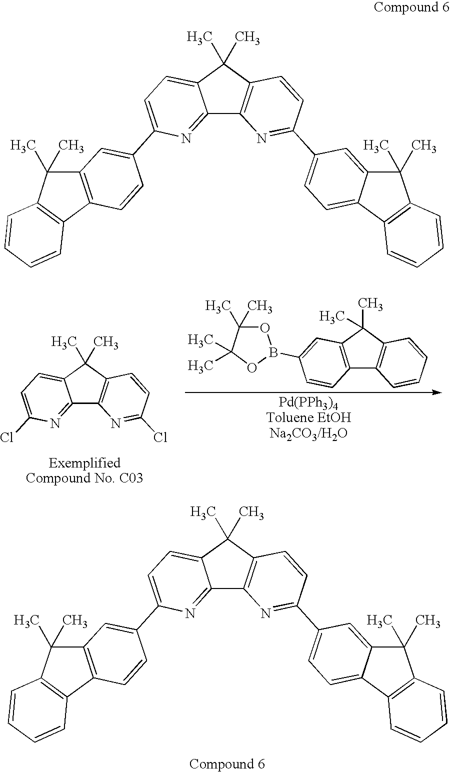

The compounds described below and solvents were sequentially added to a 3 L reactor under a nitrogen stream.

Exemplified compound No. C03: 25 g (94.3 mmol)

2-(9,9-dimethyl-9H-fluorene-2-yl)-4,4,5,5-tetramethyl-1,3,2-dioxaborolane: 66.4 g (207 mmol)

Toluene: 750 mL

Ethanol: 250 mL

10% sodium carbonate aqueous solution: 250 mL

Tetrakis triphenylphosphine palladium: 5.45 g (4.71 mmol)

Next, this reaction solution was stirred with heating under reflux for 6 hours. Thereafter, 100 mL of an aqueous 10% sodium carbonate solution and 5.45 g (4.71 mmol) of tetrakis triphenylphosphine palladium were added. The resultant was further stirred with heating under reflux for 5 hours, cooled to room temperature, and then stirred overnight. The precipitate was filtered, and sequentially washed with water, and then acetone. The obtained solid matter was dissolved in 1150 mL of chloroform. The chloroform solution was purified by column chromatography (silica gel: 1.5 kg, mobile phase: toluene), and washed with toluene to remove slurry, thereby obtaining 34.0 g of a white crystalline compound 6.

579.29 which is M+ of this compound was confirmed by matrix-assisted laser desorption-ionization time-of-flight mass spectrometry (MALDI-TOF MS).

¹H-NMR(CDCl₃):6(ppm)=8.35(d,2H,J=1.2 Hz), 8.14(dd,2H,J1=8.0,J2=1.6 Hz),7.91(d,2H,J=8.0 Hz),7.85(d,2 H,J=7.2 Hz),7.78(dd,2H,J1=6.4,J2=2.4 Hz),7.48(dd,2H,J1=6.4,J2=1.6 Hz),7.39-7.32(m,4H),1.62(s,6H),1.60(s,12H)

### SYNTHESIS EXAMPLE 2

10 g of the following compound 8 was synthesized in the same manner as in Synthesis Example 1, except that in Synthesis Example 1, the boronic acid ester represented by Formula of compound 7 was used in place of the 2-(9,9-dimethyl-9H-fluorene-2-yl)-4,4,5,5-tetramethyl-1,3,2-dioxaborolane.

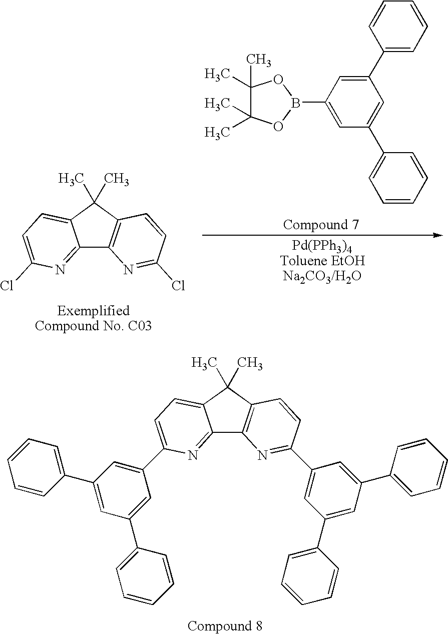

¹H-NMR(CDCl₃):δ(ppm)=8.40(d,4H,J=1.8 Hz), 7.93(d,2H,J=8.2 Hz),7.90(s,2H),7.88(dd,4H,J1=3.4,J2=1.6 Hz), 7.78(t,4H,J=1.8 Hz),7.76(t,4H,J=2.3 Hz),7.49-7.45(m,8H),7.39(tt,4H,J1=7.0,J2=1.5 Hz),1.55(s,6H)

652.29 which is M+ of this compound was confirmed by matrix-assisted laser desorption-ionization time-of-flight mass spectrometry (MALDI-TOF MS).

In Synthesis Example 1, various organic boronic acids or organic boronic acid esters shown in the left column of Tables 1 to 3 were used in place of the 2-(9,9-dimethyl-9H-fluorene-2-yl)-4,4,5,5-tetramethyl-1,3,2-dioxaborolane. Except this respect, a diazafluorene derivative shown in the right column of Tables 1 to 3 can be synthesized in the same manner as in Synthesis Example 1.

TABLE 1

| Organic boronic acid or | |
|---|---|
| Organic boronic acid ester | Diazafluorene derivative |
| 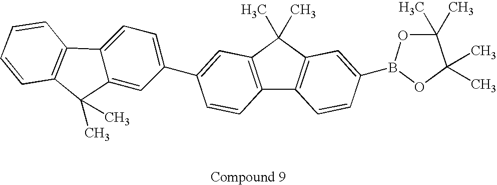 | 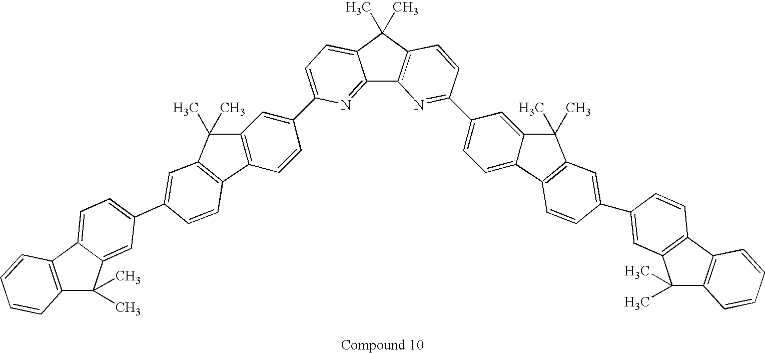 |
| 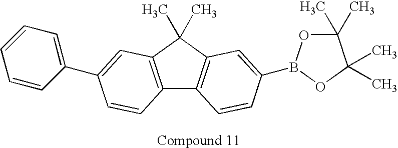 | 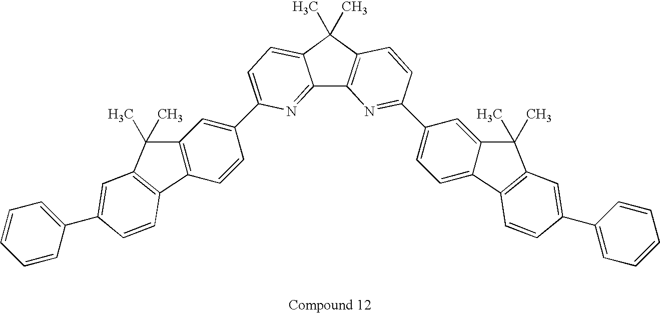 |
| 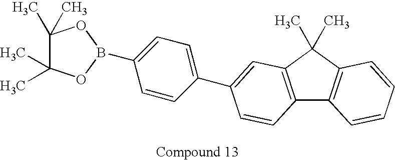 | 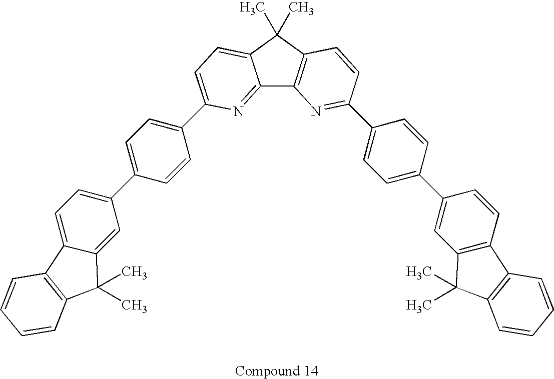 |
| 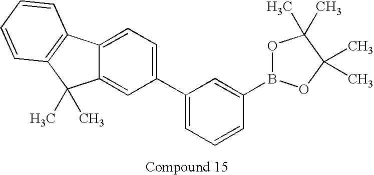 | 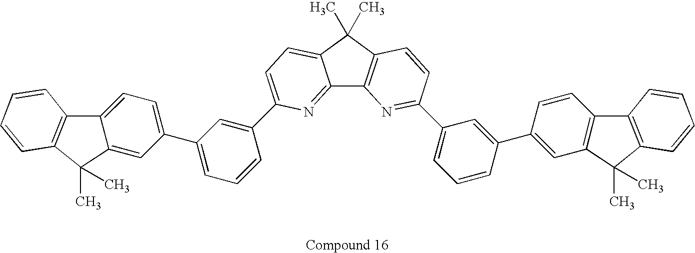 |
| 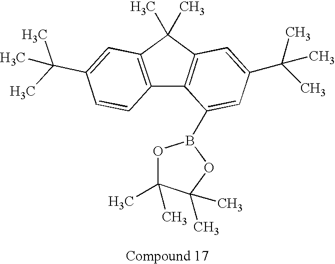 | 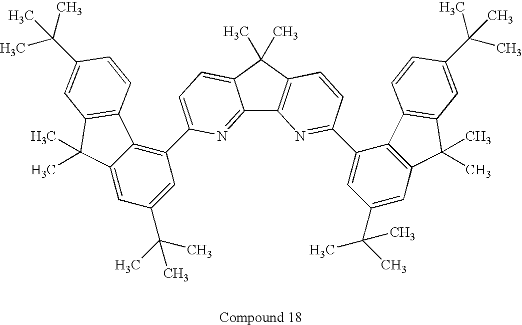 |
| 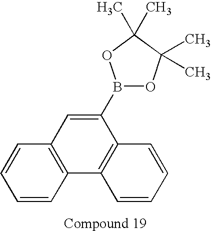 | 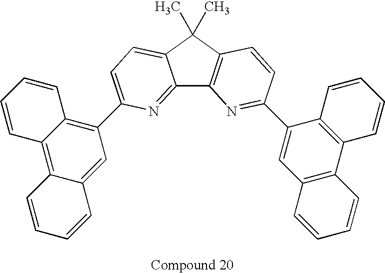 |

TABLE 2

| Organic boronic acid or | |
|---|---|
| Organic boronic acid ester | Diazafluorene derivative |
| 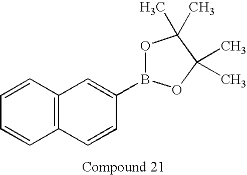 | 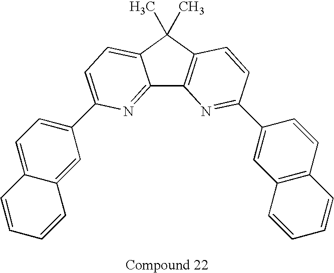 |
| 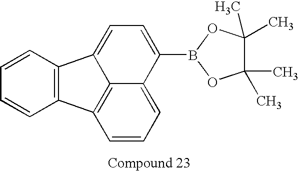 | 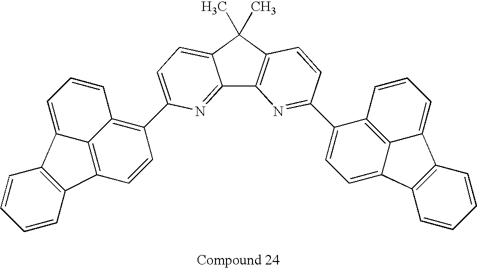 |
| 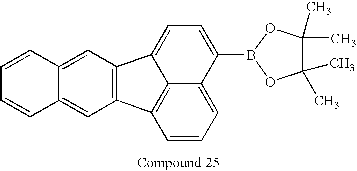 | 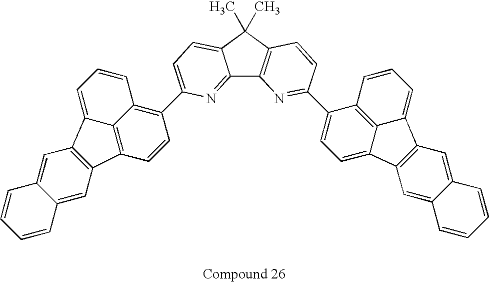 |
| 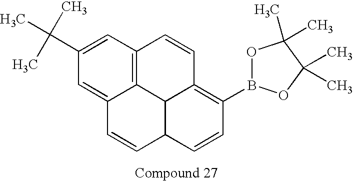 | 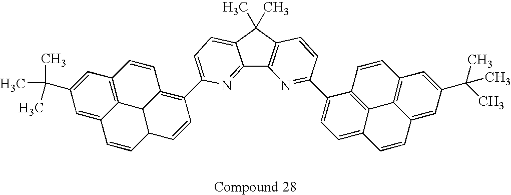 |
| 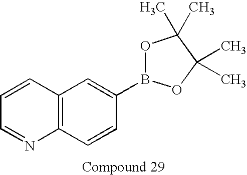 | 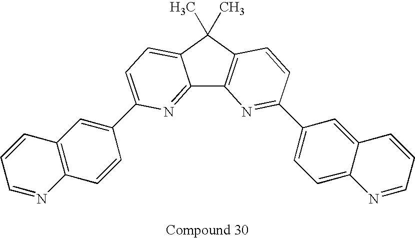 |
| 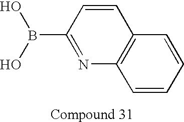 | 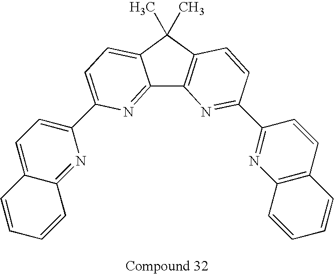 |

TABLE 3

| Organic boronic acid or | |
|---|---|
| Organic boronic acid ester | Diazafluorene derivative |
| 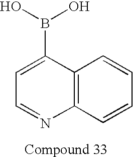 | 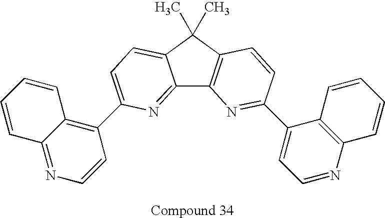 |
| 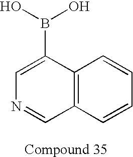 | 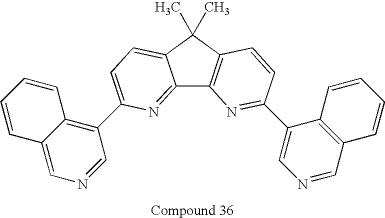 |
| 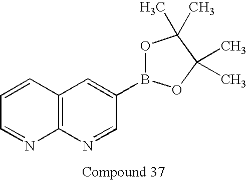 | 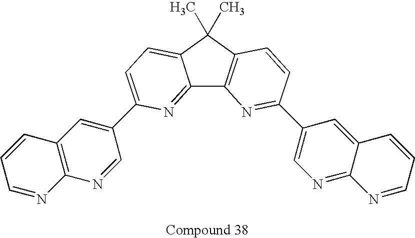 |

Further, in Synthesis Example 1, by following the method of Synthesis Example 1 except using the exemplified compounds shown in the left column of Tables 4 and 5 in place exemplified compound No. C03, diazafluorene derivatives shown in the right column of Tables 4 and 5 can be synthesized.

TABLE 4

| Exemplified | |
|---|---|
| Compound No. | Diazafluorene derivative |
| C01 | 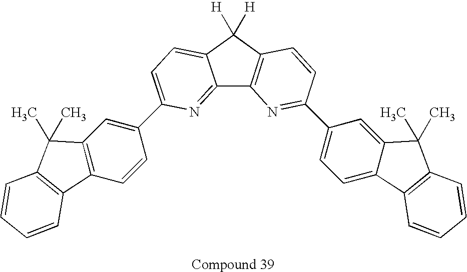 |
| C02 | 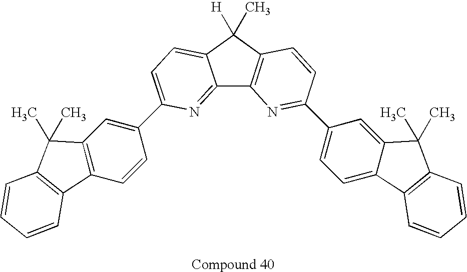 |
| C04 | 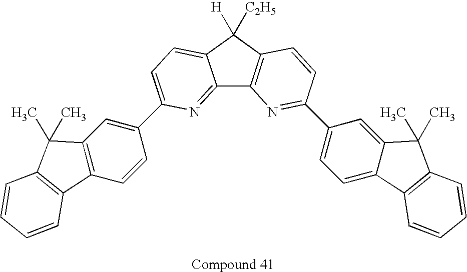 |
| C05 | 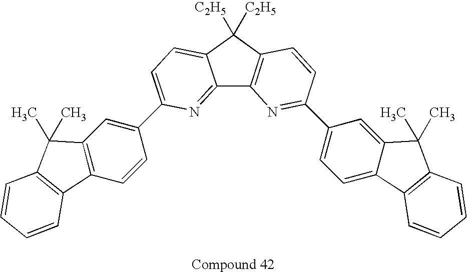 |
| C12 | 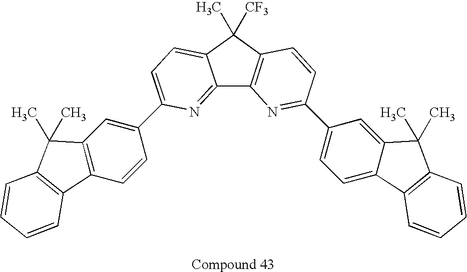 |

TABLE 5

| Exemplified | |
|---|---|
| Compound No. | Diazafluorene derivative |
| B15 | 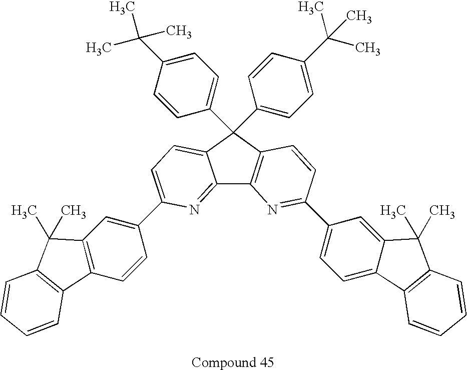 |
| B16 | 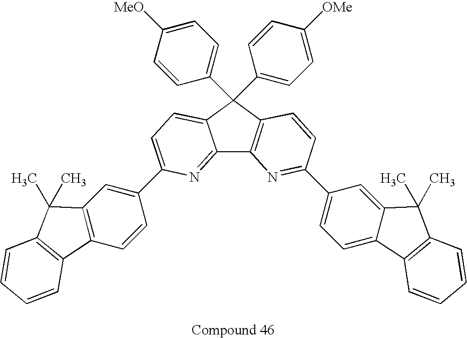 |
| I17 | 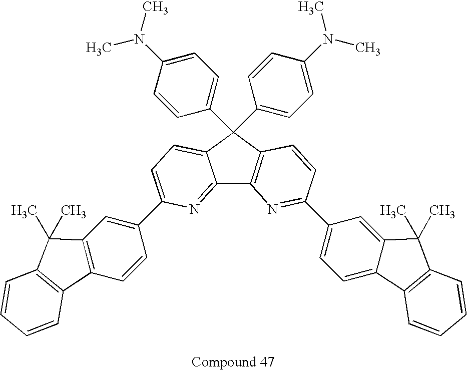 |

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2006-351758, filed Dec. 27, 2006, which is hereby incorporated by reference herein in its entirety.

What is claimed is:

1. A diazafluorene compound represented by the general formula (1): 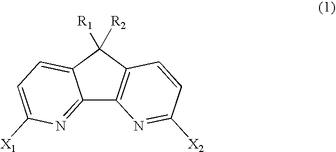
wherein R₁ and R₂ each represent a hydrogen atom, a substituted or unsubstituted alkyl group, or a substituted or unsubstituted aryl group, and may be the same or different from each other; and X₁ and X₂ each represent a halogen atom, and may be the same or different from each other.
